(19) United States
(12) Patent Application Publication
(10) Pub. No.: US 2023/0355444 A1
(43) Pub. Date: Nov. 9, 2023

(54) CHEST WALL NEGATIVE PRESSURE SUPPORT DEVICE

(71) Applicants: Yingchun HU, Luzhou City, Sichuan (CN); Qian ZHANG, Luzhou City, Sichuan (CN)

(72) Inventors: Yingchun HU, Luzhou City, Sichuan (CN); Qian ZHANG, Luzhou City, Sichuan (CN); Muhu CHEN, Luzhou City, Sichuan (CN)

(21) Appl. No.: 18/246,828

(22) PCT Filed: Aug. 6, 2021
(86) PCT No.: PCT/CN2021/111136

§ 371 Date: Mar. 27, 2023

(30) Foreign Application Priority Data
Sep. 28, 2020 (CN) 202011039820.3

(51) Int. Cl. A61F 13/00; A61H 9/00; A61F 13/14; A61M 1/00

(52) U.S. Cl. CPC A61F 13/00068; A61H 9/0057; A61F 13/143; A61M 1/912; A61H 2205/084

(57) ABSTRACT

A chest wall negative pressure support device, comprising a plastic support body (1) attached to the outer side of a chest wall in a sealed mode. A flow guide groove (2) is formed in the middle of the side surface of the plastic support body (1) in contact with the outer side of the chest wall, and a negative pressure aspirator (3) is provided on the side surface of the plastic support body (1) away from the outer side of the chest wall. An air inlet of the negative pressure aspirator (3) is communicated with the flow guide groove (2) by means of a negative pressure air outlet (4) formed in the plastic support body (1), and an exhaust port of the negative pressure aspirator (3) is communicated with an exhaust pipe (5). The plastic support body (1) is tightly attached to the outer side of the chest wall in a sealed mode, the plastic support body (1) forms an outer bracket on the outer side of the chest wall to simulate ribs for supporting, swing of a mediastinum is prevented, and the process of an on-site treatment operation is simple. In addition, the chest cavity of a patient cannot be compressed, secondary injury to the patient cannot be caused, and the pain of the patient is greatly relieved.

### TECHNICAL FIELD

The present invention relates to the technical field of medical structures, and particularly relates to a chest wall negative pressure support device.

### BACKGROUND ART

“Flail chest”, also known as multiple fractures of multiple ribs, is a severe disease of acute chest trauma. Its mortality rate is about 25%, and the main fatal factor is mediastinal swing.

The existing first aid methods for “flail chest” include cotton pad packing, skin/external rib traction and tracheal intubation. However, the cotton pad packing easily causes chest compression of a patient, skin/external rib traction is difficult during operation and easily makes the patient suffer from severe pain, and it is also hard to perform tracheal intubation for field first aid.

### SUMMARY

In view of the shortcomings existing in the prior art, the present invention provides a chest wall negative pressure support device to solve the problems in the prior art including complex filed first aid for flail chest, and much likeliness to cause chest compression or severe pain of the patient.

In order to achieve the above objective, the present invention adopts the following technical solution: a chest wall negative pressure support device, including a plastic support body attached to the outer side of a chest wall in a sealed mode, where a flow guide groove is formed in the middle of the side surface of the plastic support body in contact with the outer side of the chest wall, a negative pressure aspirator is provided on the side surface of the plastic support body away from the outer side of the chest wall, an air inlet of the negative pressure aspirator is communicated with the flow guide groove by means of a negative pressure air outlet formed in the plastic support body, and an exhaust port of the negative pressure aspirator is communicated with an exhaust pipe.

Further, the plastic support body includes an adherent dressing layer, a first sponge cushion layer, a plastic metal mesh layer, a second sponge cushion layer, and a contactable adhesive film layer that are sequentially overlapped and fixedly connected, where the adherent dressing layer and the outer side of the chest wall are sealedly attached.

Further, the flow guide groove includes a strip-shaped main flow guide groove portion with one end communicated with the negative pressure air outlet, where both ends of each side of the strip-shaped main flow guide groove portion are communicated by a plurality of branch flow guide groove portions, and the plurality of branch flow guide groove portions on each side of the strip-shaped main flow guide groove portion are sequentially arranged at intervals outside the strip-shaped main flow guide groove portion.

Further, an inner cross drainage tube is arranged in the strip-shaped main flow guide groove portion in the length direction, one end of the inner cross drainage tube is communicated with the negative pressure air outlet, and the other end thereof is communicated with the end portion of the branch flow guide groove portion far away from the negative pressure air outlet.

Further, the side surface of the plastic support body away from the outer side of the chest wall is fixedly provided with a plastic fixing sleeve with an open end, and the bottom of the negative pressure aspirator is embedded in the plastic fixing sleeve and then fixed by an adjustable fastener connected between the negative pressure aspirator and the plastic fixing sleeve.

Further, the adjustable fastener includes a plastic top block fixedly arranged at the bottom of the plastic fixing sleeve, an annular lock groove formed on the inner wall of the plastic fixing sleeve along the circumferential direction thereof, and a plurality of rigid fastening balls annularly arranged on the outer wall of the negative pressure aspirator, where the outer wall of the negative pressure aspirator is further hinged with two locking structures arranged symmetrically with the negative pressure aspirator as the center, an adjustable traction structure on the upper part of the outer wall of the negative pressure aspirator is connected between the two locking structures, and the adjustable traction structure controls the movement of the two locking structures toward or away from each other in a rotating manner; after the bottom of the negative pressure aspirator is embedded in the plastic fixing sleeve, the bottom of the negative pressure aspirator is abutted against the plastic top block, the locking ends of the two locking structures all rotate into the annular lock groove to abut against each other, and all the plurality of rigid fastening balls are abutted against the inner wall at the opening of the plastic fixing sleeve

Further, the outer wall of the negative pressure aspirator is further fixedly sleeved with an annular airbag, the annular airbag is communicated with a suction adjustment mechanism that controls its suction or inflation state, and the suction adjustment mechanism is adjusted based on the rotating manner of the adjustable traction structure; after the bottom of the negative pressure aspirator is embedded in the plastic fixing sleeve, the annular airbag is sandwiched between the negative pressure aspirator and the inner wall of the plastic fixing sleeve, and when the two locking structures move toward each other and are abutted against the annular lock groove, the annular airbag is deflated.

Further, each of the locking structures includes a locking rod with one end hinged with the outer wall of the negative pressure aspirator, and a locking roller rotatably connected with the other end of the locking rod, where a tension spring is arranged between the middle of the locking rod and the outer wall of the negative pressure aspirator, the locking rod is arranged obliquely upward, and the adjustable traction structure is communicated with the middle of the locking rod.

Further, the adjustable traction structure includes a wire take-up rod that is rotatably arranged on the upper part of the outer wall of the negative pressure aspirator and two guide rings fixedly arranged on the lower part of the outer wall of the negative pressure aspirator, where each guide ring is slidably provided with two traction wires, one end of each of the two traction wires is fixedly wound on the wire take-up rod, and the other end thereof is communicated with the middle of the locking rods in the two locking structures in a one-to-one correspondence.

Further, the suction adjustment mechanism includes a sealed piston sleeve fixedly arranged on the upper part of the outer wall of the negative pressure aspirator, and a traction rod rotatably connected at the eccentric position of the end portion of the wire take-up rod, where the sealed piston sleeve is slidably provided with a piston block, one side of the piston block is fixedly connected with an adjusting rod that freely penetrates from the sealed piston sleeve, the end of the adjusting rod arranged outside the sealed piston sleeve is rotatably and slidably connected with the end of the traction rod away from the wire take-up rod, and the side of the sealed piston sleeve away from the adjusting rod is communicated with the annular airbag through a communication pipe.

Compared with the prior art, the present invention has the following beneficial effects:

The plastic support body is attached to the outer side of the chest wall, the negative pressure aspirator is started to draw the air or liquid in the flow guide groove from the negative pressure air outlet into the negative pressure aspirator and then discharge it from the exhaust pipe, and negative pressure is formed between the plastic support body and the outer side of the chest wall, so that the plastic support body is tightly attached to the outer side of the chest wall in a sealed mode, the plastic support body forms an outer bracket on the outer side of the chest wall to simulate ribs for supporting, swing of a mediastinum is prevented, and the process of an on-site treatment operation is simple. In addition, the chest cavity of a patient cannot be compressed, secondary injury to the patient cannot be caused, and the pain of the patient is greatly relieved.

### BRIEF DESCRIPTION OF THE DRAWINGS

FIG. 1 is a front view of the present invention in use;

FIG. 2 is a reverse view of the present invention in use;

FIG. 3 is an A-A sectional diagram in FIG. 2; and

FIG. 4 is an enlarged view of a part B in FIG. 1.

### DETAILED DESCRIPTION OF THE EMBODIMENTS

The present invention will be further described in detail below with reference to the specific implementations:

Reference numerals include: plastic support body 1, flow guide groove 2, negative pressure aspirator 3, negative pressure air outlet 4, exhaust pipe 5, adherent dressing layer 101, first sponge cushion layer 102, plastic metal mesh layer 103, second sponge cushion layer 104, contactable adhesive film layer 105, strip-shaped main flow guide groove portion 201, branch flow guide groove portion 202, plastic fixing sleeve 6, plastic top block 7, annular lock groove 8, rigid fastening ball 9, annular airbag 10, locking rod 11, locking roller 12, tension spring 13, wire take-up rod 14, guide ring 15, traction wire 16, sealed piston sleeve 17, traction rod 18, piston block 19, adjusting rod 20, communication pipe 21.

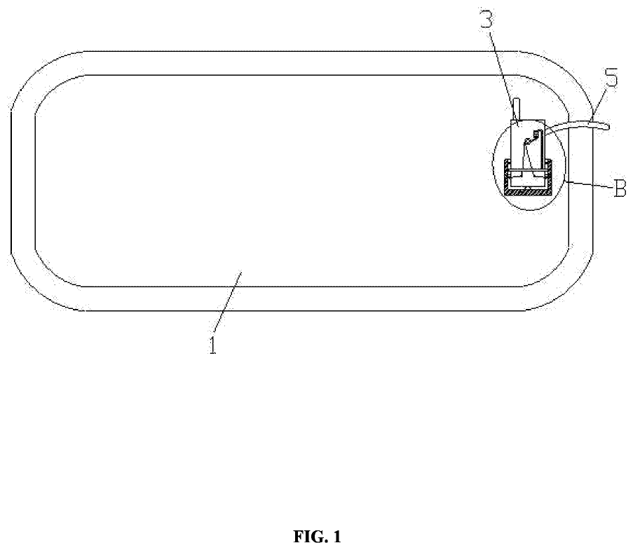

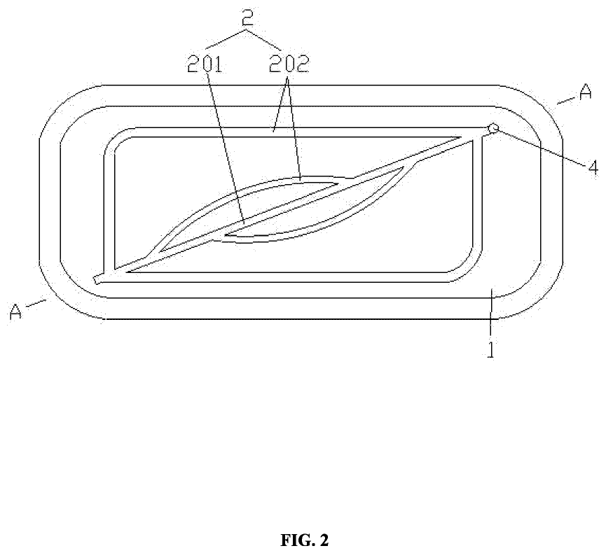

In combination with FIG. 1 and FIG. 2, the present invention provides a chest wall negative pressure support device, including a plastic support body 1 attached to the outer side of a chest wall in a sealed mode, where a flow guide groove 2 is formed in the middle of the side surface of the plastic support body 1 in contact with the outer side of the chest wall, a negative pressure aspirator 3 is provided on the side surface of the plastic support body 1 away from the outer side of the chest wall, an air inlet of the negative pressure aspirator 3 is communicated with the flow guide groove 2 by means of a negative pressure air outlet 4 formed in the plastic support body, and an exhaust port of the negative pressure aspirator 3 is communicated with an exhaust pipe 5; the negative pressure aspirator 3 may be a negative pressure pump with a power supply.

The plastic support body 1 is attached to the outer side of the chest wall, the negative pressure aspirator 3 is started to draw the air or liquid in the flow guide groove 2 from the negative pressure air outlet 4 into the negative pressure aspirator 3 and then discharge it from the exhaust pipe 5, and negative pressure is formed between the plastic support body 1 and the outer side of the chest wall, so that the plastic support body 1 is tightly attached to the outer side of the chest wall in a sealed mode, the plastic support body 1 forms an outer bracket on the outer side of the chest wall to simulate ribs for supporting, swing of a mediastinum is prevented, and the process of an on-site treatment operation is simple. In addition, the chest cavity of a patient cannot be compressed, secondary injury to the patient cannot be caused, and the pain of the patient is greatly relieved.

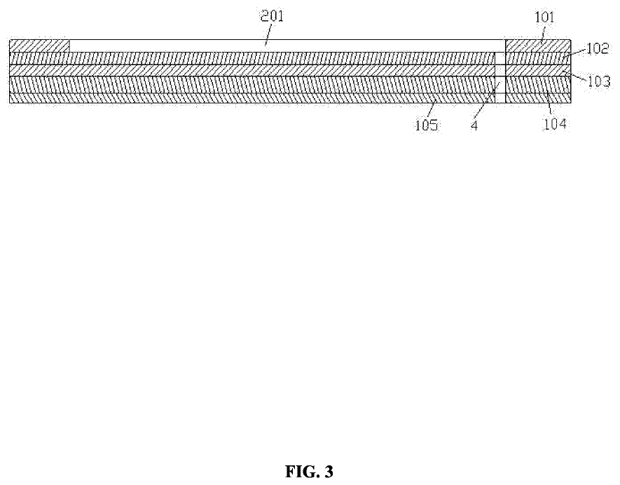

As shown in FIG. 3, according to another embodiment of the present invention, the chest wall negative pressure support device further includes an optimization of the structure of the plastic support body 1, and the adopted plastic support body 1 can be tabulate with its shape changed based on the shape of the chest wall, where the plastic support body 1 includes an adherent dressing layer 101, a first sponge cushion layer 102, a plastic metal mesh layer 103, a second sponge cushion layer 104, and a contactable adhesive film layer 105 that are sequentially overlapped and fixedly connected, where the adherent dressing layer 101 and the outer side of the chest wall are sealedly attached.

The adherent dressing layer 101 is composed of an adherent dressing that may be exposed to the skin, the plastic metal mesh layer 103 is a support frame of the plastic support body 1, and the plastic metal mesh layer 103 may be a mesh structure processed from plastic aluminum, which may be reshaped based on the shape of the chest wall and has a certain support strength; the first sponge cushion layer 102 and the second sponge cushion layer 104 are both made of sponge, have a certain cushioning performance and form a single-sided glue that is bonded and connected; the contactable adhesive film layer 105 is made of adhesive films and may be pasted on the skin so that the plastic support body 1 may be attached to the outer side of the chest wall.

As shown in FIG. 2, according to another embodiment of the present invention, the chest wall negative pressure support device further includes an optimization of the structure of the flow guide groove 2. The adopted flow guide groove 2 includes a strip-shaped main flow guide groove portion 201 with one end communicated with the negative pressure air outlet 4, where both ends of each side of the strip-shaped main flow guide groove portion 201 are communicated by a plurality of branch flow guide groove portions 202, and the plurality of branch flow guide groove portions 202 on each side of the strip-shaped main flow guide groove portion 201 are sequentially arranged at intervals outside the strip-shaped main flow guide groove portion 201.

In the embodiment, the number of branch flow guide groove portions 202 on each side of the strip-shaped main flow guide groove portion 201 is two, the distribution of the strip-shaped main flow guide groove portions 201 and the branch flow guide groove portions 202 is to ensure the negative pressure distribution area between the adherent dressing layer 101 and the outer side of the chest wall is more reasonable when the negative pressure aspirator 3 is activated to generate negative pressure in the flow guide groove 2, so that the plastic support body 1 may be closely attached to the outer side of the chest wall so as to support the outer side of the chest wall.

An inner cross drainage tube (not shown in the accompanying drawings) is arranged in the strip-shaped main flow guide groove portion 201 in the length direction, one end of the inner cross drainage tube is communicated with the negative pressure air outlet 4, and the other end thereof is communicated with the end portion of the branch flow guide groove portion 202 far away from the negative pressure air outlet 4; the arrangement of the inner cross drainage tube is to enable the air or pressure in the end portion of the branch flow guide groove portion 202 away from the negative pressure air outlet 4 to be quickly transferred into the negative pressure air outlet 4 through the inner cross drainage tube, so that the negative pressure suction in the flow guide groove 2 is more rapid.

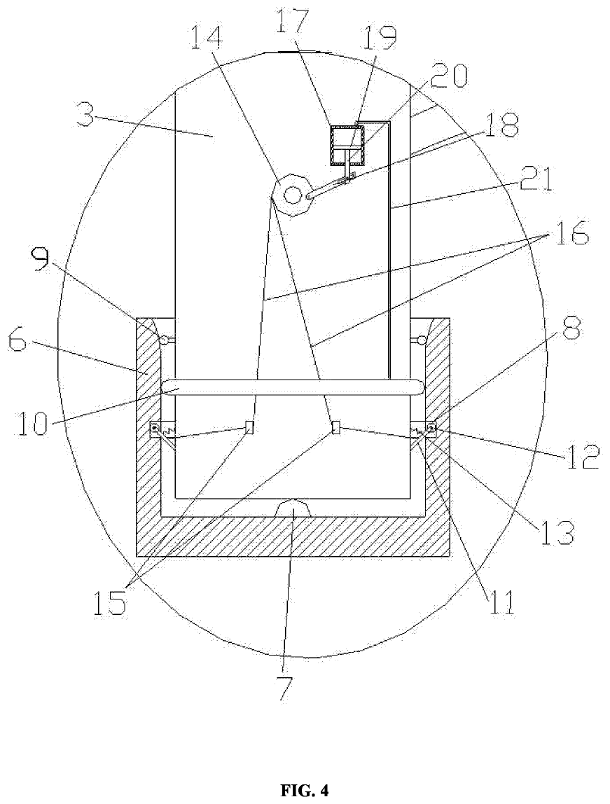

As shown in FIG. 4, according to another embodiment of the present invention, the chest wall negative pressure support device is provided, and in order to facilitate the rapid connection or disassembly of the negative pressure aspirator 3 and the plastic support body 1, the side surface of the plastic support body 1 away from the outer side of the chest wall is fixedly provided with a plastic fixing sleeve 6 with an open end, and the bottom of the negative pressure aspirator 3 is embedded in the plastic fixing sleeve 6 and then fixed by an adjustable fastener connected between the negative pressure aspirator 3 and the plastic fixing sleeve 6; the adjustable fastener is used for fixedly connecting or disconnecting the negative pressure aspirator 3 and the plastic fixing sleeve 6 quickly, and the adjustment is convenient and fast.

The adopted adjustable fastener includes a plastic top block 7 fixedly arranged at the bottom of the plastic fixing sleeve 6, an annular lock groove 8 formed on the inner wall of the plastic fixing sleeve 6 along the circumferential direction thereof, and a plurality of rigid fastening balls 9 annularly arranged on the outer wall of the negative pressure aspirator 3, where the outer wall of the negative pressure aspirator 3 is further hinged with two locking structures arranged symmetrically with the negative pressure aspirator 3 as the center, an adjustable traction structure on the upper part of the outer wall of the negative pressure aspirator 3 is connected between the two locking structures, and the adjustable traction structure controls the movement of the two locking structures toward or away from each other in a rotating manner.

After the bottom of the negative pressure aspirator 3 is embedded into the plastic fixing sleeve 6, the bottom of the negative pressure aspirator 3 is abutted against the plastic top block 7, at this time, the rotation of the adjustable traction structure makes the two locking structures rotate apart, the locking ends of the two locking structures all rotate into the annular lock groove 8, and the locking ends of the two locking structures are abutted against the inner wall of the annular lock groove 8; at the same time, all the plurality of rigid fastening balls 9 are abutted against the inner wall at the opening of the plastic fixing sleeve 6, so that multi-point positioning and fixing between the negative pressure aspirator 3 and the plastic fixing sleeve 6 are performed, and the negative pressure aspirator 3 is firmly connected with the plastic fixing sleeve 6, so that the negative pressure aspirator 3 may be stably used; reverse rotation of the adjustable traction structure makes the two locking structures rotate toward each other, and the locking ends of the two locking structures are moved out of the annular lock groove 8, so that the bottom of the negative pressure aspirator 3 may be removed from the plastic fixing sleeve 6 in a convenient and fast manner.

As shown in FIG. 4, according to another embodiment of the present invention, the chest wall negative pressure support device, in order to further improve the stability of the negative pressure aspirator 3 when connected, further includes that the outer wall of the negative pressure aspirator 3 is further fixedly sleeved with an annular airbag 10, the annular airbag 10 is communicated with a suction adjustment mechanism that controls its suction or inflation state, and the suction adjustment mechanism is adjusted based on the rotating manner of the adjustable traction structure; after the bottom of the negative pressure aspirator 3 is embedded into the plastic fixing sleeve 6, the annular airbag 10 is sandwiched between the negative pressure aspirator 3 and the inner wall of the plastic fixing sleeve 6, the annular airbag 10 is inflated in the process that the rotation of the adjustable traction structure makes the two locking structures rotate apart, so that the annular airbag 10 is tightened between the negative pressure aspirator 3 and the inner wall of the plastic fixing sleeve 6, thereby further improving the stability of the connection between the negative pressure aspirator 3 and the plastic fixing sleeve 6, and making the negative pressure aspirator 3 more stable during operation; in the process that reverse rotation of the adjustable traction structure makes the two locking structures rotate toward each other, the annular airbag 10 is deflated until the annular airbag 10 becomes completely flat, and the annular airbag 10 breaks away from the tension and fixation between the negative pressure aspirator 3 and the inner wall of the plastic fixing sleeve 6, so that the negative pressure aspirator 3 can be quickly taken out of the plastic fixing sleeve 6.

As shown in FIG. 4, according to another embodiment of the present invention, the chest wall negative pressure support device further includes structural optimization of each locking structure, each of the adopted locking structures includes a locking rod 11 with one end hinged with the outer wall of the negative pressure aspirator 3, and a locking roller 12 rotatably connected with the other end of the locking rod 11, where a tension spring 13 is arranged between the middle of the locking rod 11 and the outer wall of the negative pressure aspirator 3, the locking rod 11 is arranged obliquely upward, and the adjustable traction structure is communicated with the middle of the locking rod 11.

The tension spring 13 is designed to further increase the countervailing force between the locking roller 12 and the inner wall of the annular lock groove 8, and the inclined arrangement of the locking rods 11 is to facilitate that, in the process that the negative pressure aspirator 3 is embedded into the plastic fixing sleeve 6, both the two locking rods 11 rotate in the direction of the negative pressure aspirator 3 to prevent the locking rods 11 from obstructing the movement of the negative pressure aspirator 3 into the plastic fixing sleeve 6; when the bottom of the negative pressure aspirator 3 is abutted against the plastic top block 7, both the two locking rods 11 rotate in a direction away from the negative pressure aspirator 3 to make the locking roller 12 abutted against the inner wall of the annular lock groove 8, so that the negative pressure aspirator 3 is locked and fixed; in the process that the locking roller 12 disengages the negative pressure aspirator 3 from the locking and fixing state, the locking roller 12 is slidably abutted against the inner wall of the annular lock groove 8, so that the locking roller 12 and the annular lock groove 8 can be quickly disconnected.

The adopted adjustable traction structure includes a wire take-up rod 14 that is rotatably arranged on the upper part of the outer wall of the negative pressure aspirator 3 and two guide rings 15 fixedly arranged on the lower part of the outer wall of the negative pressure aspirator 3, where each guide ring 15 is slidably provided with two traction wires 16, one end of each of the two traction wires 16 is fixedly wound on the wire take-up rod 14, and the other end thereof is communicated with the middle of the locking rods 11 in the two locking structures in a one-to-one correspondence.

When the wire take-up rod 14 is rotated to take up the two traction wires 16, both the two locking rods 11 rotate in the direction close to the negative pressure aspirator 3, and the two locking rods 11 rotate toward each other to remove the locking roller 12 from the annular lock groove 8, so that the negative pressure aspirator 3 may be removed from the plastic fixing sleeve 6, and the plastic fixing sleeve 6 and the negative pressure aspirator 3 are disconnected; after the bottom of the negative pressure aspirator 3 is embedded in the plastic fixing sleeve 6, the bottom of the negative pressure aspirator 3 is abutted against the plastic top block 7, the wire take-up rod 14 is reversely rotated, the two traction wires 16 are taken down through the wire take-up rod 14 to make the two locking rods 11 rotate in a direction away from the negative pressure aspirator 3, and the two locking rods 11 rotate apart to make the locking roller 12 rotate into the annular lock groove 8 so as to abut against the inner wall of the annular lock groove 8.

As shown in FIG. 4, according to another embodiment of the present invention, the chest wall negative pressure support device further includes structural optimization of the suction adjustment mechanism, and the adopted suction adjustment mechanism includes a sealed piston sleeve 17 fixedly arranged on the upper part of the outer wall of the negative pressure aspirator 3, and a traction rod 18 rotatably connected at the eccentric position of the end portion of the wire take-up rod 14, where the sealed piston sleeve 17 is slidably provided with a piston block 19, one side of the piston block 19 is fixedly connected with an adjusting rod 20 that freely penetrates from the sealed piston sleeve 17, the end of the adjusting rod 20 arranged outside the sealed piston sleeve 17 is rotatably and slidably communicated with the end of the traction rod 18 away from the wire take-up rod 14, and the side of the sealed piston sleeve 17 away from the adjusting rod 20 is communicated with the annular airbag 10 through a communication pipe 21.

When the take-up rod 14 is rotated to take up the two traction lines 16, the traction rod 18 uses the adjusting rod 20 to make the piston block 19 slide in the sealed piston sleeve 17, so that a suction force is formed in the communication pipe 21, which further results in that the gas in the annular airbag 10 is removed into the sealed piston sleeve 17, the annular airbag 10 is deflated and becomes completely flat, and the annular airbag 10 breaks away from the tension and fixation between the negative pressure aspirator 3 and the inner wall of the plastic fixing sleeve 6; when the wire take-up rod 14 is reversely rotated to take down the two traction lines 16, the traction rod 18 uses the adjusting rod 20 to make the piston block 19 reversely slide in the sealed piston sleeve 17, the piston block 19 compresses the gas in the sealed piston sleeve 17 into the communication pipe 21 and then introduces the gas into the annular airbag 10 to inflate the annular airbag 10, and the annular airbag 10 is tightened and fixed between the negative pressure aspirator 3 and the inner wall of the plastic fixing sleeve 6.

It should be noted that the above embodiments are merely intended for describing the technical solutions of the present invention rather than limiting same. Although the present invention is described in detail with reference to the above embodiments, persons of ordinary skill in the art should understand that they may still make modifications or equivalent replacements to the technical solutions of the present invention without departing from the spirit and scope of the technical solutions of the present invention, which shall fall within the scope of the claims of the present invention.

What is claimed is:

1. A chest wall negative pressure support device, comprising a plastic support body attached to the outer side of a chest wall in a sealed mode, wherein a flow guide groove is formed in the middle of the side surface of the plastic support body in contact with the outer side of the chest wall, a negative pressure aspirator is provided on the side surface of the plastic support body away from the outer side of the chest wall, an air inlet of the negative pressure aspirator is communicated with the flow guide groove by means of a negative pressure air outlet formed in the plastic support body, and an exhaust port of the negative pressure aspirator is communicated with an exhaust pipe.

2. The chest wall negative pressure support device according to claim 1, wherein the plastic support body comprises an adherent dressing layer, a first sponge cushion layer, a plastic metal mesh layer, a second sponge cushion layer, and a contactable adhesive film layer that are sequentially overlapped and fixedly connected, wherein the adherent dressing layer and the outer side of the chest wall are sealedly attached.

3. The chest wall negative pressure support device according to claim 2, wherein the flow guide groove comprises a strip-shaped main flow guide groove portion with one end communicated with the negative pressure air outlet, wherein both ends of each side of the strip-shaped main flow guide groove portion are communicated by a plurality of branch flow guide groove portions, and the plurality of branch flow guide groove portions on each side of the strip-shaped main flow guide groove portion are sequentially arranged at intervals outside the strip-shaped main flow guide groove portion.

4. The chest wall negative pressure support device according to claim 3, wherein an inner cross drainage tube is arranged in the strip-shaped main flow guide groove portion in the length direction, one end of the inner cross drainage tube is communicated with the negative pressure air outlet, and the other end thereof is communicated with the end portion of the branch flow guide groove portion far away from the negative pressure air outlet.

5. The chest wall negative pressure support device according to claim 1, wherein the side surface of the plastic support body away from the outer side of the chest wall is fixedly provided with a plastic fixing sleeve with an open end, and the bottom of the negative pressure aspirator is embedded in the plastic fixing sleeve and then fixed by an adjustable fastener connected between the negative pressure aspirator and the plastic fixing sleeve.

6. The chest wall negative pressure support device according to claim 5, wherein the adjustable fastener comprises a plastic top block fixedly arranged at the bottom of the plastic fixing sleeve, an annular lock groove formed on the inner wall of the plastic fixing sleeve along the circumferential direction thereof, and a plurality of rigid fastening balls annularly arranged on the outer wall of the negative pressure aspirator, wherein the outer wall of the negative pressure aspirator is further hinged with two locking structures arranged symmetrically with the negative pressure aspirator as the center, an adjustable traction structure on the upper part of the outer wall of the negative pressure aspirator is connected between the two locking structures, and the adjustable traction structure controls the movement of the two locking structures toward or away from each other in a rotating manner; after the bottom of the negative pressure aspirator is embedded in the plastic fixing sleeve, the bottom of the negative pressure aspirator is abutted against the plastic top block, the locking ends of the two locking structures all rotate into the annular lock groove to abut against each other, and all the plurality of rigid fastening balls are abutted against the inner wall at the opening of the plastic fixing sleeve.

7. The chest wall negative pressure support device according to claim 6, wherein the outer wall of the negative pressure aspirator is further fixedly sleeved with an annular airbag, the annular airbag is communicated with a suction adjustment mechanism that controls its suction or inflation state, and the suction adjustment mechanism is adjusted based on the rotating manner of the adjustable traction structure; after the bottom of the negative pressure aspirator is embedded in the plastic fixing sleeve, the annular airbag is sandwiched between the negative pressure aspirator and the inner wall of the plastic fixing sleeve, and when the two locking structures move toward each other and are abutted against the annular lock groove, the annular airbag is deflated.

8. The chest wall negative pressure support device according to claim 7, wherein each of the locking structures comprises a locking rod with one end hinged with the outer wall of the negative pressure aspirator, and a locking roller rotatably connected with the other end of the locking rod, wherein a tension spring is arranged between the middle of the locking rod and the outer wall of the negative pressure aspirator, the locking rod is arranged obliquely upward, and the adjustable traction structure is communicated with the middle of the locking rod.

9. The chest wall negative pressure support device according to claim 8, wherein the adjustable traction structure comprises a wire take-up rod that is rotatably arranged on the upper part of the outer wall of the negative pressure aspirator and two guide rings fixedly arranged on the lower part of the outer wall of the negative pressure aspirator, wherein each guide ring is slidably provided with two traction wires, one end of each of the two traction wires is fixedly wound on the wire take-up rod, and the other end thereof is communicated with the middle of the locking rods in the two locking structures in a one-to-one correspondence.

10. The chest wall negative pressure support device according to claim 9, wherein the suction adjustment mechanism comprises a sealed piston sleeve fixedly arranged on the upper part of the outer wall of the negative pressure aspirator, and a traction rod rotatably connected at the eccentric position of the end portion of the wire take-up rod, wherein the sealed piston sleeve is slidably provided with a piston block, one side of the piston block is fixedly connected with an adjusting rod that freely penetrates from the sealed piston sleeve, the end of the adjusting rod arranged outside the sealed piston sleeve is rotatably and slidably connected with the end of the traction rod away from the wire take-up rod, and the side of the sealed piston sleeve away from the adjusting rod is communicated with the annular airbag through a communication pipe.
